(19) United States
(12) Patent Application Publication
(10) Pub. No.: US 2012/0253328 A1
(43) Pub. Date: Oct. 4, 2012

(54) COMBINED PRESENTATION UNIT FOR REPOSABLE BATTERY OPERATED SURGICAL SYSTEM

(75) Inventors: James S. Cunningham, Boulder, CO (US); William J. Dickhans, Longmont, CO (US); Russell D. Hempstead, Lafayette, CO (US); John J. Kappus, Denver, CO (US); Duane E. Kerr, Loveland, CO (US); Eric R. Larson, Boulder, CO (US); William H. Nau, JR., Longmont, CO (US); Anthony B. Ross, Boulder, CO (US); Robert B. Stoddard, Steamboat Springs, CO (US)

(73) Assignee: TYCO HEALTHCARE GROUP LP, Mansfield, MA (US)

(21) Appl. No.: 13/436,062

(22) Filed: Mar. 30, 2012

Related U.S. Application Data
(60) Provisional application No. 61/469,631, filed on Mar. 30, 2011.

(51) Int. Cl. A61B 17/00; H02J 7/00; H01R 43/00

(52) U.S. Cl. 606/1; 29/825; 320/112

(57) ABSTRACT

A portable presentation unit for sterilizing, charging, and testing components of a cordless surgical instrument is disclosed including a battery charger unit configured to recharge a battery pack of the cordless surgical instrument, a testing unit configured to test at least one of the battery pack or a reusable transducer/generator unit of the cordless surgical instrument, and a sterilization unit configured to sterilize at least one of the battery pack or the transducer/generator unit.

### CROSS REFERENCE TO RELATED APPLICATION

The present application claims the benefit of and priority to U.S. Provisional Application Ser. No. 61/469,631, filed on Mar. 30, 2011, entitled “COMBINED PRESENTATION UNIT FOR REPOSABLE BATTERY OPERATED SURGICAL SYSTEM”, the entirety of which is hereby incorporated by reference herein for all purposes.

### BACKGROUND

1. Technical Field

The present disclosure is directed to systems and methods for low-temperature sterilization, and more specifically, to low-temperature sterilization of battery packs, reusable transducer/generator units, and other components of cordless surgical instruments.

2. Background of Related Art

Portable surgical instruments are known in the medical arts. Portable surgical instruments overcome some of the drawbacks that are typically associated with surgical instruments that draw power from electrical outlets. That is, outlet driven surgical instruments utilize power cords that may create tripping and/or entanglement hazards in an operating room environment.

Typically, the portable surgical instrument includes a battery or battery assembly that is configured to removably couple or “latch” to the portable surgical instrument. In an ideal scenario, the battery or battery assembly remains coupled or “latched” to the portable surgical instrument during the entirety of the surgical procedure. However, in certain instances, the battery or battery assembly has to be uncoupled or “unlatched” from the portable surgical instrument during the surgical procedure. As can be appreciated, removable batteries or battery assemblies that are configured to quickly and easily couple or latch to a handpiece of the portable surgical instrument may prove advantageous in the surgical environment. For example, the battery or battery assembly may have to be unlatched from the surgical instrument for sterilization (or re-sterilization), charging (or recharging), replacement, etc.

Because the battery and/or TAG are reusable, sterilization protocols must be followed. While it is possible to re-sterilize a rechargeable battery or TAG using an autoclave, this high temperature method is above the temperature limits of currently available rechargeable battery cells and/or TAG components, and may lead to premature failure of a battery, TAG, or other system component.

### SUMMARY

In one aspect, disclosed is a portable surgical system consisting of a sterile, disposable handpiece unit that accepts a reusable battery pack and a reusable transducer/generator unit (TAG).

A portable presentation unit for sterilizing, charging, and testing components of a cordless surgical instrument is disclosed. The disclosed portable presentation unit includes including a battery charger unit configured to recharge a battery pack of the cordless surgical instrument, a testing unit configured to test at least one of the battery pack or a reusable transducer/generator unit of the cordless surgical instrument, and a sterilization unit configured to sterilize at least one of the battery pack or the transducer/generator unit.

In an aspect of the present disclosure, the sterilization unit is configured to sterilize at least one of the battery pack or the transducer/generator unit using a low temperature sterilization method.

In an aspect of the present disclosure, the low temperature sterilization method is selected from the group consisting of ultraviolet radiation, vapor hydrogen peroxide system, ozone, and silver nano-particles.

In an aspect of the present disclosure, the battery charger unit is configured to test the battery pack.

In an aspect of the present disclosure, the portable presentation unit further includes a controller in operative communication with at least one of the battery charger unit, testing unit, or sterilization unit and configured to control at least one of the battery charger unit, testing unit, or sterilization unit.

In an aspect of the present disclosure, the portable presentation unit includes a user interface in operative communication with the controller and configured to command the controller to control, in response to a user input, the at least one of the battery charger unit, testing unit, or sterilization unit.

In an aspect of the present disclosure, the controller is configured to control, in response to the user input, at least one of the battery charger unit, testing unit, or sterilization unit to perform at least one of testing the battery pack, charging the battery pack, testing the transducer/generator unit, recharging the battery pack, sterilizing the battery pack, or sterilizing the transducer/generator unit.

In an aspect of the present disclosure, the user interface includes at least one of a display, an indicator, and an annunciator for indicating a status of the portable presentation unit.

In an aspect of the present disclosure, the status includes at least one of a sterilization state, a battery charge level, and a test result.

In an aspect of the present disclosure, the controller is configured to detect a supply level of a sterilization consumable and to cause an indicator on the user interface to be displayed when a sterilization consumable needs replenishment.

A surgical system is disclosed that includes a battery pack, a transducer/generator unit, a cordless surgical instrument configured to accept at least one of the battery pack and the transducer/generator unit, and a presentation unit. The presentation unit includes a battery charger unit configured to recharge the battery pack, a testing unit configured to test at least one of the battery pack and the transducer/generator unit and a sterilization unit configured to sterilize at least one of the battery pack and the transducer/generator unit.

In an aspect of the present disclosure, the sterilization unit is configured to sterilize at least one of the battery pack or the transducer/generator unit using a low temperature sterilization method.

In an aspect of the present disclosure, the low temperature sterilization method is selected from the group consisting of ultraviolet radiation, vapor hydrogen peroxide system, ethylene oxide, ozone, and silver nano-particles.

In an aspect of the present disclosure, the battery charger unit is configured to test the reusable battery pack.

In an aspect of the present disclosure, the presentation unit further includes a controller in operative communication with at least one of the battery charger unit, testing unit, or sterilization unit and configured to control at least one of the battery charger unit, testing unit, or sterilization unit.

In an aspect of the present disclosure, the presentation unit includes a user interface in operative communication with the controller and configured to command the controller, in response to a user input, to control the at least one of the battery charger unit, testing unit, or sterilization unit.

In an aspect of the present disclosure, the controller is configured to control, in response to the user input, at least one of the battery charger unit, testing unit, or sterilization unit to perform at least one of testing the battery pack, charging the battery pack, testing the transducer/generator unit, recharging the battery pack, sterilizing the battery pack and sterilizing the transducer/generator unit.

A method for charging, testing, and sterilizing components of a cordless surgical instrument is disclosed. The method includes the steps of providing a presentation unit including a battery charger unit, a testing unit and a sterilization unit, inserting a battery pack of the cordless surgical instrument into the presentation unit to engage the battery charger unit, inserting a transducer/generator unit of the cordless surgical instrument into the presentation unit to engage the testing unit, and manipulating a user interface of the presentation unit to activate at least one of the battery charger unit, testing unit, or sterilization unit to perform at least one of a charging cycle, a testing cycle, or a sterilization cycle.

In an aspect of the present disclosure, upon completion of the at least one of the charging cycle, testing cycle, or sterilization cycle, the user interface provides an indication of a completed cycle.

In an aspect of the present disclosure, the method further comprises removing at least one of the battery pack or the transducer/generator unit from the presentation device and inserting the at least one of the battery pack or the transducer/generator unit into the cordless surgical instrument, and performing a surgical procedure.

It is contemplated that any of the above aspects and embodiments may be combined without departing from the scope of the present disclosure.

### BRIEF DESCRIPTION OF DRAWINGS

The accompanying drawings, which are incorporated in and constitute a part of this specification, illustrate embodiments of the disclosure and, together with a general description of the disclosure given above and the detailed description of the embodiments given below, serve to explain the principles of the disclosure, wherein:

FIG. 1 is a perspective view of an embodiment of a cordless surgical instrument in accordance with the present disclosure;

FIG. 2 is a side, cross-sectional view of the cordless surgical instrument of FIG. 1;

FIG. 3 is a perspective view of another embodiment of a cordless surgical instrument in accordance with the present disclosure;

FIG. 4 is a side, cross-sectional view of the cordless surgical instrument of FIG. 3;

FIG. 5 is a perspective view of an embodiment of a presentation unit in accordance the present disclosure;

FIG. 6 is a perspective view of the presentation unit of FIG. 5 illustrating the user interface of the presentation unit disposed on the cover, with the cover in a closed position;

FIG. 7 is a schematic view of the components of the presentation unit of FIG. 5; and

FIG. 8 is an illustrative view of the components of the presentation unit of FIG. 5.

### DETAILED DESCRIPTION OF EMBODIMENTS

Embodiments of the presently disclosed system will now be described in detail with reference to the drawing figures wherein like reference numerals identify similar or identical elements. As used herein and as is traditional, the term “distal” refers to that portion which is furthest from the user while the term “proximal” refers to that portion which is closest to the user.

The present disclosure is directed to systems and methods for low-temperature sterilization of a battery, battery packs, reusable transducer/generator units (TAGs), and other components of a cordless device for ultrasonic or radiofrequency (RF) surgical instruments. It is contemplated that the system of the present disclosure may be used for other types of cordless medical instruments including, for example, thermal, microwave, or other similar electrosurgical instruments.

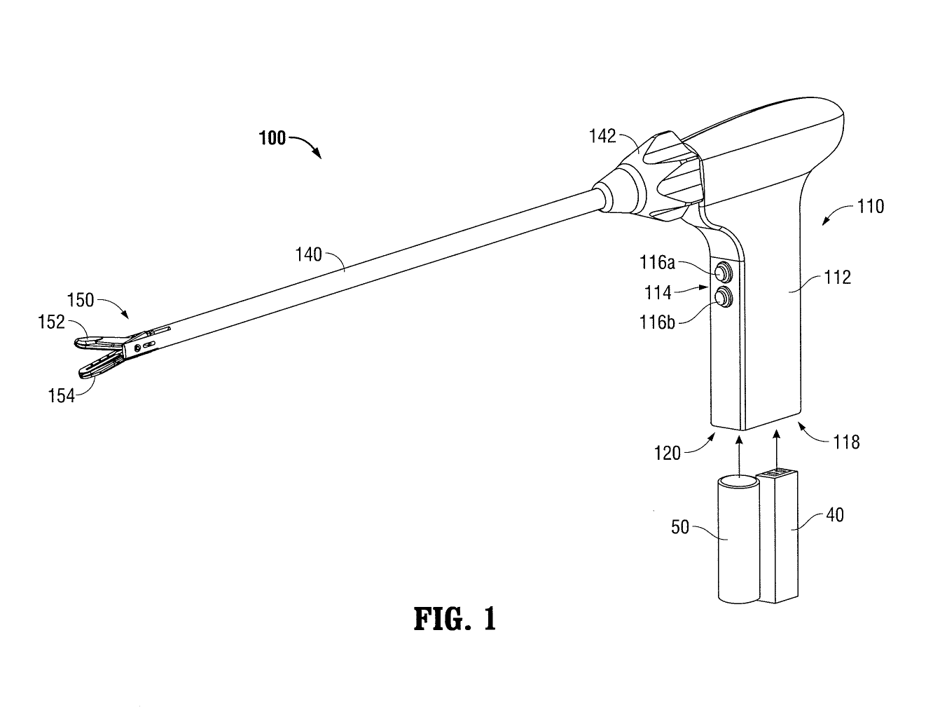

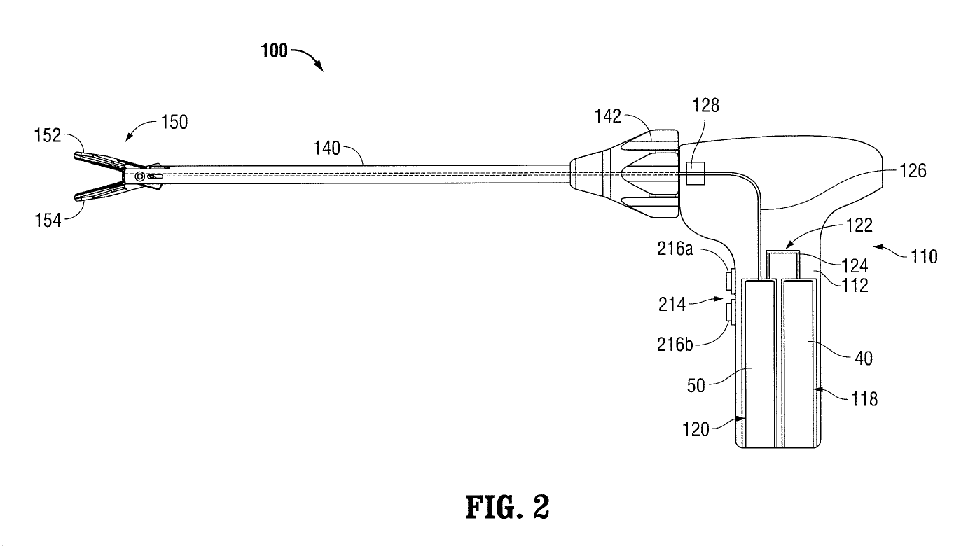

With reference now to FIGS. 1 and 2, there is disclosed a cordless surgical instrument 100 including a handle 110, an elongated shaft 140 extending from handle 110 and an end effector 150 at a distal end of elongate shaft 140. Handle 110 includes a grip 112 having activation elements 114 disposed thereon, for example, buttons or switches 116a, 166b. Elongated shaft 140 extends distally from handle 110 and may include a rotation mechanism 142 configured to rotate elongated shaft 140 and end effector 150 relative to handle 110. End effector 150 includes a pair of jaws 152, 154 that are transitionable between a first, open configuration, where jaw members 152, 154 are configured to receive tissue therebetween, and a second configuration, where jaw members 152, 154 are approximated to grasp the tissue disposed therebetween. An example of a suitable cordless surgical instrument is disclosed in co-pending U.S. patent applicant Ser. No. 13,312,299, filed on Dec. 6, 2011, entitled “PORTABLE SURGICAL INSTRUMENTS”, the entirety of which is hereby incorporated by reference herein for all purposes.

With reference now to FIG. 2, handle 110 further includes a first slot 118 that is configured to selectively receive a battery pack 40 and a second slot 120 that is configured to selectively receive a TAG 50. The first and second slots 118, 120 are in electrical communication via an electrical connection element 122, for example, a wire 124. This allows electrical energy from battery pack 40 to be supplied to TAG 50 during use. A transmission element 126 couples to the tag 50 and powers a waveguide 128 which energizes one or both jaw members 152 and 154. Activation elements e.g. 116a and 116b are configured to enable a user to control operation of the device, for example without limitation, opening and/or closing jaw members 152 and 154, activating and/or deactivating delivery of energy from the TAG to jaw members 152 and 154, a waveguide 128, and/or end effector 150.

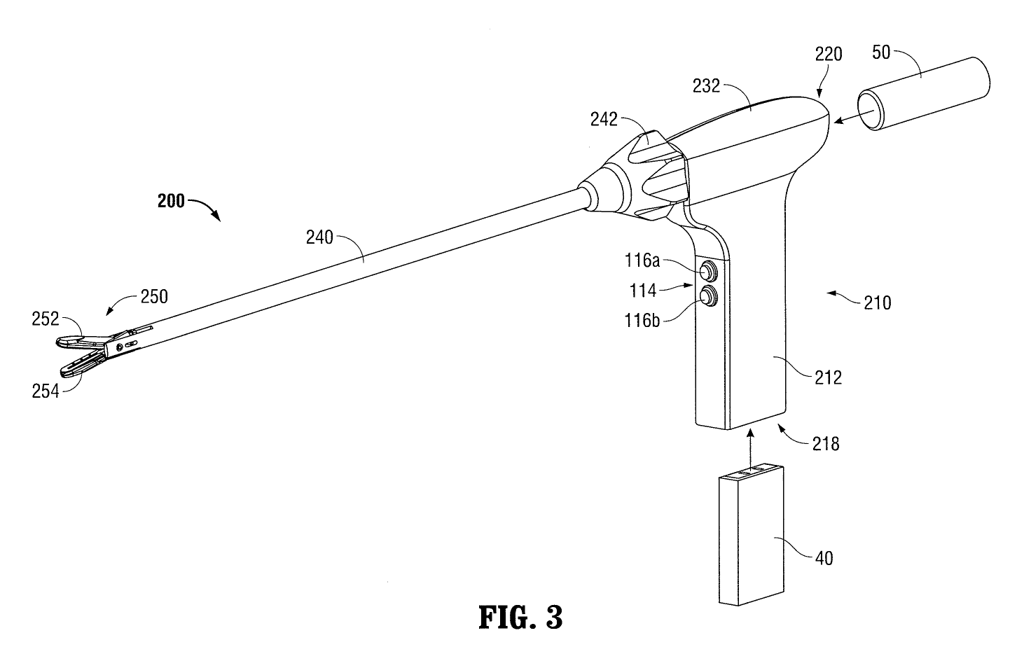

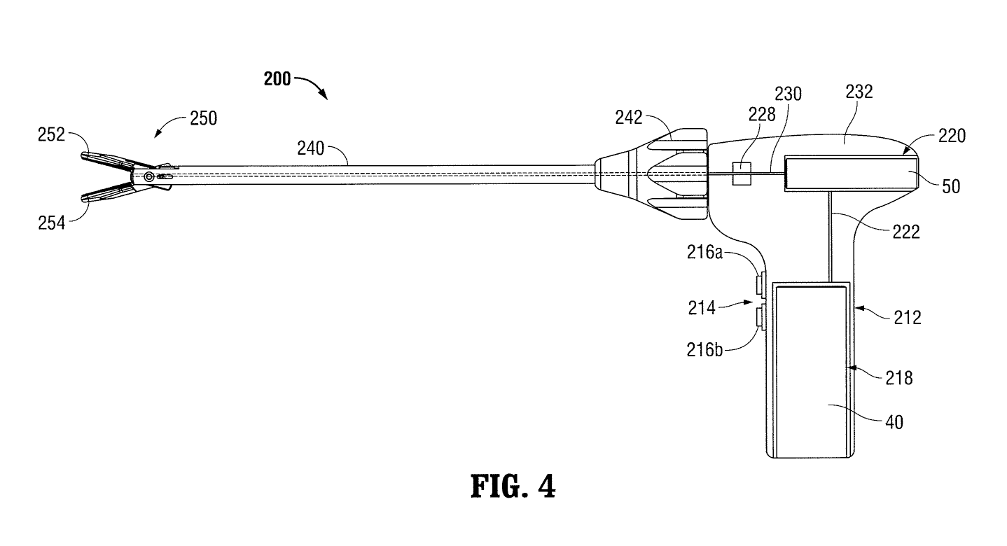

With reference to FIGS. 3 and 4, another surgical instrument 200 is disclosed which is similar to surgical instrument 100 and will be described only with respect to the differences found therebetween. As illustrated in FIG. 4, surgical instrument 200 includes handle 210, an elongate shaft 240 extending from the handle 210 and an end effector 250 at a distal end of the elongate shaft 240. Handle 210 includes a grip 212 and an upper portion 232. Grip 212 includes a first slot 218 configured to selectively receive battery pack 40 and upper portion 232 includes a second slot 220 configured to selectively receive of TAG 50. The first and second slots 218, 220 are electrically connected together via an electrical connection element 222, for example, a wire. A transmission element 226 (for example, a waveguide 228 or a wire 230, depending on the type of surgical instrument) extends from second slot 220 through elongate shaft 240 to end effector 150 for transmitting energy from TAG 50 to end effector 250. Activation elements e.g. 216a and 216b are configured to enable a user to control operation of the device, for example without limitation, opening and/or closing jaw members 252 and 254, activating and/or deactivating delivery of energy from the TAG to jaw members 252 and 254, a waveguide 228, and/or to end effector 250.

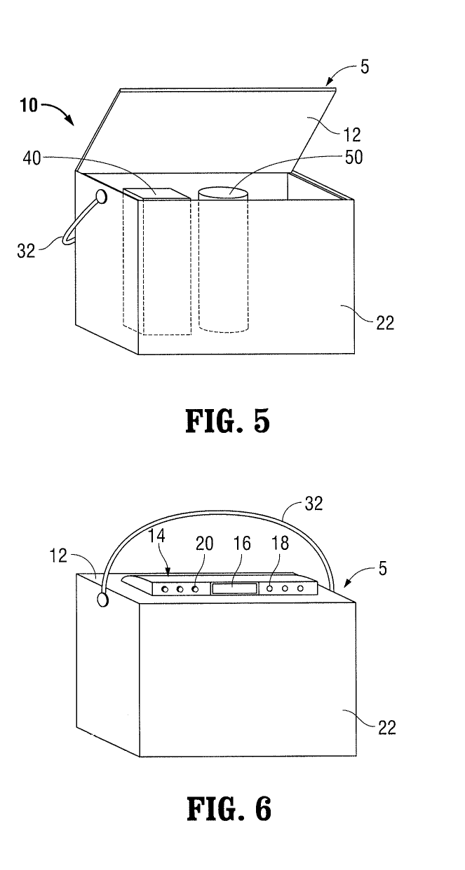

With reference now to FIGS. 5-8, a sterilization system 5 is disclosed that incorporates sterilization, recharging, and storage functions into a storage/surgical presentation unit 10. The presentation unit 10 includes a battery charging unit 26, a test unit 28, and a sterilization unit 30. Presentation unit 10 may also act as a sterilization storage unit.

Presentation unit 10 includes a cover 12, a container 22 and a handle 32. Cover 12 includes a user interface 14 disposed thereon including, for example, display panels 16, indicators/annunciators 18, and input elements 20. In embodiments, user interface 14 may output visual information relating to the status, e.g. a sterilization state, a battery charge level, or a test result, of presentation unit 10 via display panel 16 and/or indicators/annunciators 18 or may make an audio indication, such as, for example, a beep or tone. Input elements 20 may include, for example, knobs, switches, buttons, touch panels or other similar elements 20 which enable a user to activate, deactivate, and monitor a function of the presentation unit.

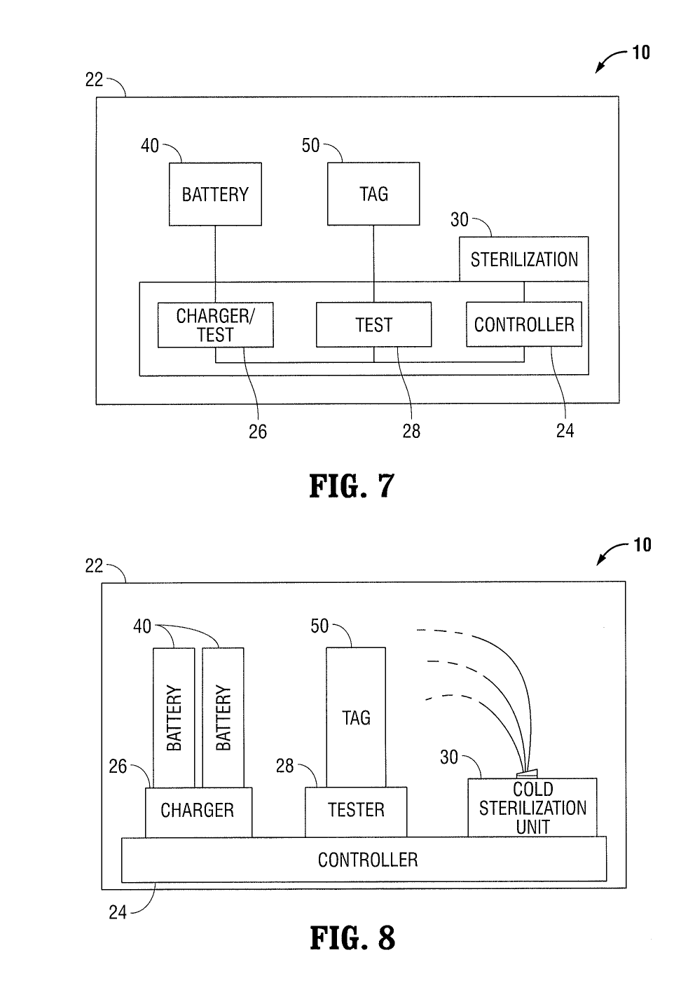

With reference now to FIGS. 7 and 8, container 22 includes a controller 24 a battery charging unit 26, a testing unit 28 and a sterilization unit 30 disposed therein. Controller 24 is in operative communication with user interface 14, battery charging unit 26, testing unit 28 and sterilization unit 30 to control the charging, testing, and sterilization cycles of the presentation unit 10 based on a user input at user interface 14. Controller 24 may be programmed to, for example and without limitation, execute a method of testing battery pack 40, charging battery pack 40, testing TAG 50, recharging battery pack 40, and/or sterilizing battery pack 40 and/or TAG 50. Controller 24 may additionally or alternatively be configured to sense or detect a supply level of a sterilization consumable and to cause an indicator on user interface 14 to be displayed when a sterilization consumable needs replenishment (e.g., refill or replace a hydrogen peroxide reservoir, refill or replace a silver nano-particle reservoir, check ozone generator electrodes, and the like).

Battery charging unit 26 is configured to receive battery pack 40 and includes circuitry for recharging battery pack 40. Battery charging unit 26 is also configured to test battery pack 40 to ensure that sufficient charge is available for performing the surgical procedure and/or to monitor the health of the battery pack. It is contemplated that additional battery packs 40 may be received by battery charging unit 26 such that multiple battery packs 40 may be recharged, tested and sterilized simultaneously. This allows the surgeon to change battery packs 40 as needed during the surgical procedure without leaving the sterile space. It is contemplated that testing unit 28 may additionally or alternatively be configured to test battery pack 40, as described below.

Testing unit 28 is configured to receive TAG 50 and to test TAG 50 to ensure proper operation. For example, testing unit 28 may execute a diagnostic test on TAG 50 to ensure that TAG 50 is operating within acceptable parameters for the surgical procedure. It is contemplated that additional TAGs 50 may be received by testing unit 28 such that multiple TAGs 50 may be tested and sterilized at the same time.

Sterilization unit 30 is configured to sterilize the interior of container 22 including any battery packs 40 or TAGs 50 that are received therein. Sterilization unit 30 may sterilize battery pack 40 and TAG 50 by using cold sterilization to preserve the life expectancy of battery pack 40 and TAG 50, for example, UV radiation, a small scale vapor hydrogen peroxide system, ozone, silver nano-particles, or other methods of dry, low temperature sterilization. During sterilization, cover 12 is closed to provide and maintain a sterile sealed environment within container 22. This enables sterilization to occur within container 22 and maintains the interior of container 22 as a sterile environment until cover 12 is re-opened.

During use, the battery pack 40 and TAG 50 are sterilized within presentation unit 10 prior to the surgical procedure and presentation unit 10 is then transported to the sterile environment of the operating room (“OR”) with sterilized battery pack 40 and TAG 50 inside are ready for use. It is contemplated that presentation unit 10 could alternatively be transported to the OR prior to sterilization where sterilization is performed prior to the surgical procedure.

In the OR, a technician or circulating nurse may plug the presentation unit 10 into a wall outlet to charge, test, or maintain the battery pack 40 and TAG 50. Prior to any surgery, OR personnel may initiate testing using user interface 14, and if the tests are positive, the circulating nurse could open cover 12 of presentation unit 10 and present the sterile battery pack 40 and TAG 50 to the scrub nurse or surgeon for assembly with the cordless surgical instrument in the sterile field.

After surgery, all reusable components of the system may be transported to central processing for cleaning, re-sterilization, and charging using presentation unit 10.

A sterilization system in accordance with the present disclosure has many benefits. The system allows for small scale re-sterilization of the reusable parts of the system without having to rely on a third party industrial sterilization method that may not be available in all hospitals. The system also eliminates the extra handling and storage space required by separate sterilization, battery charging, and testing units, and further, eliminates the need to inventory multiple servicing units. Having a single presentation unit simplifies the workflow of the reusable components both before and after the surgical procedure, and gives hospital personnel additional flexibility to charge, test, and sterilize components directly prior to, or even during, the surgical procedure.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to the precise embodiments described herein, and that various other changes and modifications may be effected by one skilled in the art without departing from the scope or spirit of the present disclosure.

What is claimed is:

1. A portable presentation unit for a surgical instrument, comprising:
a battery charger unit configured to recharge a battery pack of the surgical instrument; and at least one of:
a testing unit configured to test at least one of the battery pack or a reusable transducer/generator unit of the surgical instrument; and
a sterilization unit configured to sterilize at least one of the battery pack or the transducer/generator unit.

2. The portable presentation unit according to claim 1, wherein the sterilization unit is configured to sterilize at least one of the battery pack or the transducer/generator unit using a low temperature sterilization method.

3. The portable presentation unit according to claim 2, wherein the low temperature sterilization method is selected from the group consisting of ultraviolet radiation, vapor hydrogen peroxide system, ethylene oxide, ozone, and silver nano-particles.

4. The portable presentation unit according to claim 1, wherein the battery charger unit includes the testing unit.

5. The portable presentation unit according to claim 1, wherein the portable presentation unit further includes a controller in operative communication with at least one of the battery charger unit, testing unit, or sterilization unit, the controller being configured to control at least one of the battery charger unit, testing unit, or sterilization unit.

6. The portable presentation unit according to claim 5, wherein the portable presentation unit includes a user interface in operative communication with the controller and configured to command the controller to control, in response to a user input, at least one of the battery charger unit, testing unit, or sterilization unit.

7. The portable presentation unit according to claim 6, wherein the user interface includes at least one of a display, an indicator, and an annunciator for indicating a status of the portable presentation unit.

8. The portable presentation unit according to claim 7, wherein the status includes at least one of a battery charge level, a test result, and a sterilization state.

9. The portable presentation unit according to claim 6, wherein the controller is configured to detect a supply level of a sterilization consumable and to cause an indicator on the user interface to be displayed when a sterilization consumable needs replenishment.

10. A surgical system, comprising:
a battery pack;
a transducer/generator unit;
a cordless surgical instrument configured to accept at least one of the battery pack and the transducer/generator unit; and
a presentation unit, comprising:
a battery charger unit configured to recharge the battery pack;
a testing unit configured to test at least one of the battery pack and the transducer/generator unit; and
a sterilization unit configured to sterilize at least one of the battery pack and the transducer/generator unit.

11. The surgical system according to claim 10, wherein the sterilization unit is configured to sterilize at least one of the battery pack or the transducer/generator unit using a low temperature sterilization method.

12. The surgical system according to claim 11, wherein the low temperature sterilization method is selected from the group consisting of ultraviolet radiation, vapor hydrogen peroxide system, ethylene oxide, ozone, and silver nano-particles.

13. The surgical system according to claim 10, wherein the battery charger unit is configured to test the reusable battery pack.

14. The surgical system according to claim 10, wherein the presentation unit further includes a controller in operative communication with at least one of the battery charger unit, testing unit, or sterilization unit, the controller being configured to control at least one of the battery charger unit, testing unit or sterilization unit.

15. The surgical system according to claim 14 wherein the presentation unit includes a user interface in operative communication with the controller and configured to command the controller, in response to a user input, to control at least one of the battery charger unit, testing unit, or sterilization unit.

16. A method for charging, testing, and sterilizing components of a cordless surgical instrument, the method comprising the steps of:
providing a presentation unit including:
a battery charger unit;
a testing unit; and
a sterilization unit;
inserting a battery pack of the cordless surgical instrument into the presentation unit to engage the battery charger unit;
inserting a transducer/generator unit of the cordless surgical instrument into the presentation unit to engage the testing unit; and
manipulating a user interface of the presentation unit to activate at least one of the battery charger unit, testing unit, or sterilization unit to perform at least one of a charging cycle, a testing cycle, or a sterilization cycle.

17. The method according to claim 16, wherein upon completion of at least one of the charging cycle, testing cycle, or sterilization cycle, the user interface provides an indication of a status of the presentation unit.

18. The method according to claim 17, further comprising:
removing at least one of the battery pack or the transducer/generator unit from the presentation device and inserting the at least one of the battery pack or the transducer/generator unit into the cordless surgical instrument; and
performing a surgical procedure.
